(19) United States
(12) Patent Application Publication
(10) Pub. No.: US 2021/0353901 A1
(43) Pub. Date: Nov. 18, 2021

(54) DEMAND VALVE RESTRICTOR

(71) Applicant: Vitalograph (Ireland) Ltd., Co. Clare (IE)

(72) Inventors: Adrian JOYCE, Co. Clare (IE); Aidan BOLAND, Co. Clare (IE); Enda KELLY, Co. Clare (IE)

(73) Assignee: Vitalograph (Ireland) Ltd., Co. Clare (IE)

(21) Appl. No.: 17/282,956

(22) PCT Filed: Oct. 4, 2019
(86) PCT No.: PCT/EP2019/076945

§ 371 Date: Apr. 5, 2021

(30) Foreign Application Priority Data
Oct. 5, 2018 (EP) 18198944.3

(51) Int. Cl. A61M 16/20

(52) U.S. Cl. CPC A61M 16/20; A61M 2205/3334; A61M 2205/70

(57) ABSTRACT

A demand valve restrictor (1) co-operable with a demand valve for respiratory devices (3) comprising a damping plate (5) having a calibration opening (6) and a respiration opening (7) for controlled air flow through the restrictor (1) and a seal mechanism (4) attached to the damping plate (5) movable between a calibration setting allowing damped air flow through the calibration opening (6) and a patient setting allowing uninterrupted air flow through the respiration opening (7).

### INTRODUCTION

This invention relates to a demand valve restrictor for respiratory devices and to a demand valve comprising the restrictor.

### BACKGROUND OF THE INVENTION

Many respiratory devices such as resuscitators and pulmonary function testing equipment which supply gases to patients employ demand valves which operate to supply gas to the patient in response to the patient's inhalation demand. Generally, such devices are made up of a pressure chamber having a breathing gas inlet and a breathing gas outlet for directing gas from the pressure chamber to the outlet. More particularly, a valve is mounted in the inlet which is movable from a closed position to an open position to allow gas flow into the chamber in response to a negative pressure created in the chamber by the movement of a pressure sensitive diaphragm caused by the patient's inhalation demand.

Pulmonary function devices employing demand valves must be calibrated in order to ensure optimal performance and patient safety. In general, calibration of respiratory devices and the demand valves in the devices requires the use of calibration syringes or other respiratory simulating devices. Moreover, the American Thoracic Society (ATS)/European Respiratory Society (ERS) Technical Standards 2017 for single breath diffusion tests require that calibration syringes must be used with demand valves to check the accuracy of devices that perform single breath diffusion and the tests must return results within certain defined limits. However, it has been found that the use of a syringe can in fact compromise demand valve performance—a syringe can cause gas flow to become extremely pulsatile thus disrupting gas flow and rendering the associated volume readings extremely inaccurate.

More particularly, a calibration syringe, or other similar rigid pieces of equipment, when used with a demand valve, can create pulses of gas in a demand valve. Friction between moving parts in the equipment creates air pulses which travel along the equipment to the gas source at the demand valve. These pulses affect the diaphragm in the demand valve, causing it to flutter. Accordingly, when gas is flowing through the demand valve, this fluttering generates large pulses of gas. Pulmonary function equipment measures inspiratory and expiratory pressures for the calculation of respiratory flow rates and volumes but will misinterpret large gas pulses as high pressures and consequently high flow rates and high volumes. Accordingly, to meet the American Thoracic Society (ATS)/European Respiratory Society (ERS) Technical Standards 2017 for single breath diffusion tests, it is essential that a demand valve can perform satisfactorily with a calibration syringe but this is inherently impossible with the demand valves and associated calibration equipment of the prior art.

U.S. Pat. No. 4,121,580 describes a squeeze bag resuscitator having a proportionating valve adapted to control the proportion of oxygen in a breathing gas mixture. The proportionating valve therefore makes direct contact with breathing gases and is fitted to the resuscitator housing instead of a demand valve. Accordingly, alternatively, a demand valve as described in U.S. Pat. No. 3,473,529 can be connected to the resuscitator housing. However, the proportionating valve is not adapted to be fitted to the demand valve so that the proportionating valve and the demand valve can be simultaneously fitted to the resuscitator. Therefore, the proportionating valve is incapable of functioning as a demand valve air flow restrictor for use in calibrating a demand valve. Moreover, even if the proportioning valve were used with a demand valve with a calibration syringe as outlined above, pulsatile gas flow would still occur resulting in inaccurate volume readings.

It is therefore an object to provide an improved valve for use in a respiratory device.

### SUMMARY OF THE INVENTION

According to the invention there is provided, as set out in the appended claims, a demand valve restrictor co-operable with a demand valve for respiratory devices comprising:
a damping plate having a respiration opening and a calibration opening for controlled air flow through the restrictor and
a seal mechanism attached to the damping plate, the seal mechanism being movable between a patient setting allowing uninterrupted air flow through the respiration opening and a calibration setting allowing damped air flow through the calibration opening.

Preferably, the respiration opening is a relatively larger opening and the calibration opening is a relatively smaller opening.

Suitably, the seal mechanism comprises a controller for controlling movement of the seal mechanism between the patient setting and the calibration setting. Preferably, the controller comprises a rotatable dial mounted on the damping plate. More preferably, the rotatable dial comprises a first dial opening complementary with the respiration opening and a second dial opening complementary with the calibration opening, the first dial opening being contiguous with the respiration opening in the patient setting and the second dial opening being contiguous with the calibration opening in the calibration setting.

Advantageously, the controller further comprises a lug on the damping plate engageable in a slot on the rotatable dial.

Suitably, the slot comprises keepers to hold the lug at the patient and calibration settings.

Preferably, the demand valve restrictor as claimed further comprises an assembly nut mounted between the damping plate and the dial.

Suitably, the demand valve restrictor further comprises a securing mechanism between the damping plate and the assembly nut to secure the assembly nut to the damping plate. Preferably, the securing mechanism comprises a key-like securing mechanism. More preferably, the key-like securing mechanism comprises a catch on the damping plate and a complementary catch on the assembly nut.

Optionally, the securing mechanism comprises at least one finger on the damping plate and at least one corresponding finger slot on the assembly nut.

Preferably, the dial comprises a securing mechanism engageable with the assembly nut to rotatably attach the dial to the assembly nut.

Suitably, the demand valve restrictor further comprises seals between the damping plate and the seal mechanism.

Preferably, the demand valve restrictor comprises a demand valve mounting for mounting the restrictor on a demand valve flowhead. More preferably, the demand valve mounting is provided on the damping plate.

The invention also extends to a respiratory device demand valve comprising a demand valve restrictor as hereinbefore defined.

In general, the invention therefore provides a demand valve air-flow restrictor for respiratory devices comprising:

a damping plate having a respiration opening and a calibration opening for controlled air flow through the restrictor and a seal mechanism attached to the damping plate, the seal mechanism being movable between a patient setting allowing uninterrupted air flow through the respiration opening and a calibration setting allowing damped air flow through the calibration opening.

The demand valve air flow restrictor of the invention is adapted to co-operate with and to be used simultaneously with a demand valve to improve the performance of the demand valve i.e. the demand valve restrictor does not replace demand valves but serves to positively augment demand valves. More particularly, the demand valve air flow restrictor when mounted on the demand valve, e.g. via the demand valve mounting on the damping plate, allows for accurate calibration of respiratory devices using the calibration setting by ensuring a damped airflow into the atmospheric side of the demand valve to reduce the pitch of pulses when using a syringe. Conversely, in the patient setting, uninterrupted air flow occurs through the restrictor to allow the diaphragm in the demand valve to operate freely so that a patient can breathe on demand in comfort.

Moreover, unlike the proportionating valve of the prior art, the demand valve air flow restrictor of the invention does not contact the breathing gases but instead controls the atmospheric side of the diaphragm inside a demand valve, thereby improving the performance of the demand valve with calibration equipment.

### BRIEF DESCRIPTION OF THE DRAWINGS

The invention will now be described, by way of example only, with reference to the accompanying drawings in which:

FIG. 1 is a perspective view from above and one side of a demand valve restrictor assembly attached to a demand valve;

FIG. 2 is a perspective view from the rotatable seal plate or dial end of the demand valve restrictor of FIG. 1;

FIG. 3 is an exploded view of the demand valve restrictor separated from the demand valve;

FIG. 4 is an isometric view of the damping plate of the demand valve restrictor from the assembly nut side of the damping plate;

FIG. 5 is a reverse isometric view of the damping plate from the demand valve side of the damping plate;

FIG. 6 is a plan view of the demand valve side of the damping plate;

FIG. 7 is a plan view of the assembly nut/dial side of the damping plate;

FIG. 8 is a cross-sectional view along the line VIII-VIII of FIG. 7;

FIG. 9 is an isometric view of the rotatable seal plate or dial of the seal mechanism of the demand valve restrictor;

FIG. 10 is a reverse isometric view of the rotatable dial of the seal mechanism from the demand valve side of the dial;

FIG. 11 is a plan view of the demand valve face of the rotatable dial;

FIG. 12 is a plan view of the opposite end face of the rotatable dial;

FIG. 13 is a cross-sectional view along the line XIII-XIII of FIG. 12;

FIG. 14 is an isometric view of the assembly nut of the demand valve restrictor from the dial side of the assembly nut;

FIG. 15 is a reverse isometric view of the assembly nut from the demand valve side of the assembly nut;

FIG. 16 is a plan view of the demand valve face of the assembly nut;

FIG. 17 is a side view of the assembly nut;

FIG. 18 is a cross-sectional view along the line XVIII-XVIII of FIG. 17;

FIG. 19 is a cross-sectional view along the line XIX-XIX of FIG. 17, and

FIG. 20 is an exploded view of the demand valve restrictor mounted on a demand valve flowhead of a respiratory device.

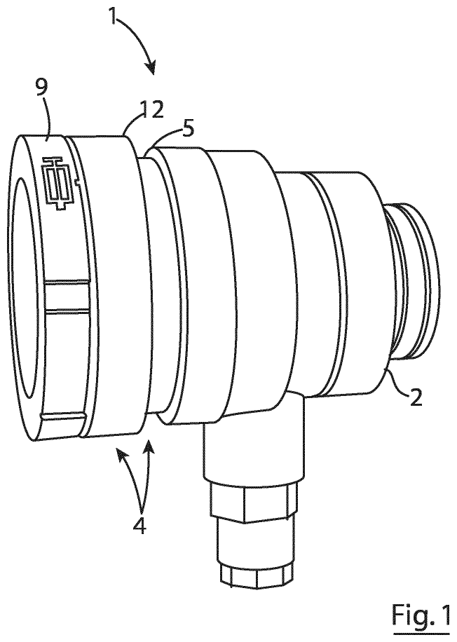

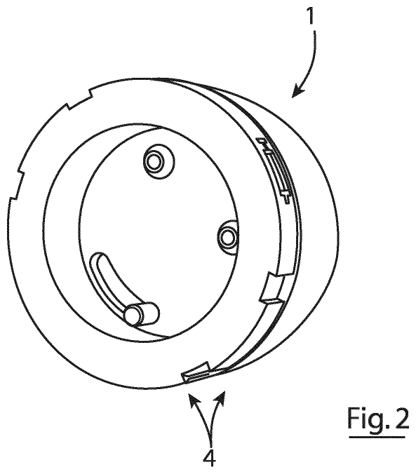

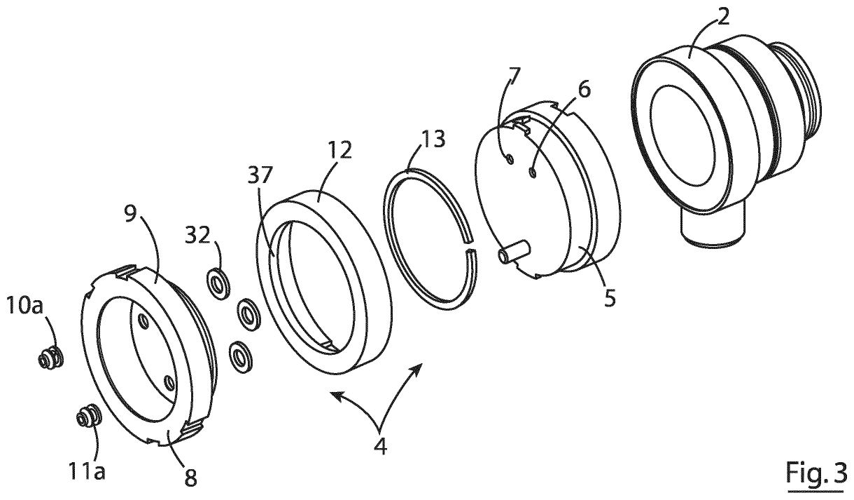

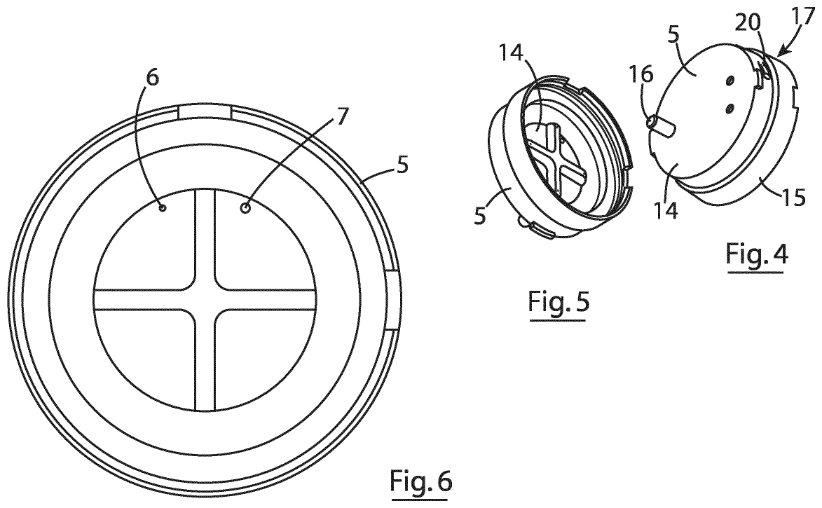

### DETAILED DESCRIPTION OF THE INVENTION

As shown in the drawings, an air flow restrictor 1 for a demand valve 2 in respiratory devices 3 is generally made up of a damping plate 5, attachable to the demand valve 2, for controlling air flow through a seal mechanism 4. The damping plate 5 has a first relatively smaller calibration opening 6 and a second relatively larger respiration opening 7 arcuately spaced apart from the calibration opening 6 and both gaseously communicable with an atmospheric side of the demand valve 2 while the seal mechanism 4 has a controller 8 for selectively controlling movement of air through the calibration and respiration openings 6,7 by rotatably moving the seal mechanism 4 between a patient setting for uninterrupted gas flow through the demand valve 2 and a calibration setting for damped gas flow through the demand valve 2 to dampen pulses when calibrating the device using a calibration syringe. The controller 8 is in the form of a manually rotatable dial 9 on the restrictor 1 movable between the patient and calibration settings having first and second selectable arcuately spaced apart dial openings 10,11 selectively complementary with the calibration and respiration openings 6,7 in the damping plate 5. The spaced apart dial openings 10, 11 can be provided with seals 10a, 11a. The manually rotatable dial 9 is secured to the damping plate 5 by an intermediate lockable assembly nut 12 between the rotatable dial 9 and the damping plate 5 while seals 10a, 11a ensure an airtight fit of the seal mechanism 4 on the damping plate 5 so that air flow through the restrictor 1 occurs via the calibration and respiration openings 6,7 and the first and second selectable dial openings 10,11 only as required. Only one of the dial openings 10, 11 is in use at any given time, so ring seals 32 provide an airtight seal around the dial opening that is not in use.

The diameter and length of the calibration opening 6 is optimised to prevent pulsatile gas flow from the demand valve 2. The diameter of the calibration opening can be in the range from 0.13 mm to 0.33 mm. The ratio of the diameter of the calibration opening to the diameter of the respiration opening is approximately 1:5. The length of the calibration opening can be in the range from 5.90 mm to 6.10 mm.

A snap ring 13 holds the manually rotatable dial 9 securely to the assembly nut 12, whilst still allowing the dial to be freely rotated. The snap ring 13 fits between a groove on the dial 9 and a shoulder on the assembly nut 12.

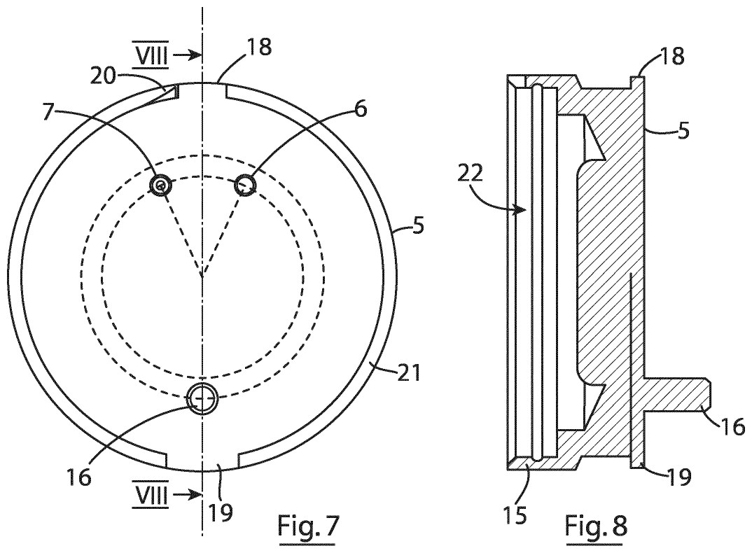

As shown particularly in FIGS. 6 to 8, the damping plate 5 is made up of a circular damping plate wall 14 defining the spaced apart calibration and respiration openings 6, 7 and a skirt-like damping plate sidewall 15 depending from the damping plate wall 14. The damping plate wall 14 is provided with an outwardly projecting lug 16 which forms part of the controller 8 of the seal mechanism 4.

The damping plate 5 is further provided with a key-like securing mechanism 17 for securing the damping plate 5 to the lockable assembly nut 12. The securing mechanism 17 is made up of oppositely disposed outwardly projecting peripheral first and second catch-like fingers 18,19 on the damping plate wall 14 and a catch 20 on the damping plate sidewall 15.

The damping plate sidewall 15 is contoured to define a shoulder 21 for receiving the lockable assembly nut 12 which is secured in place by the catch 20. This shall be explained more fully below.

Internally, the demand valve air-flow restrictor 1 and more particularly the damping plate sidewall 15 of the demand valve air-flow restrictor 1, is provided with a demand valve mounting 22 for attaching the damping plate and hence the restrictor 1 to the demand valve 2.

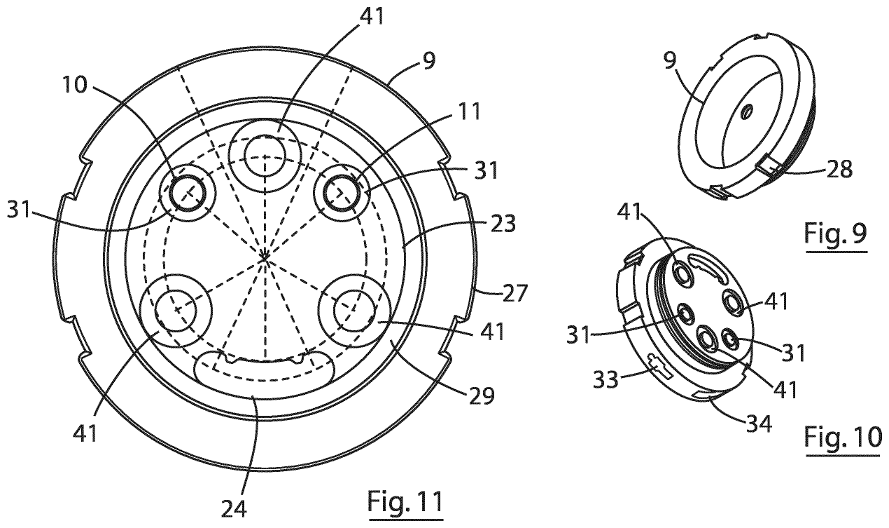

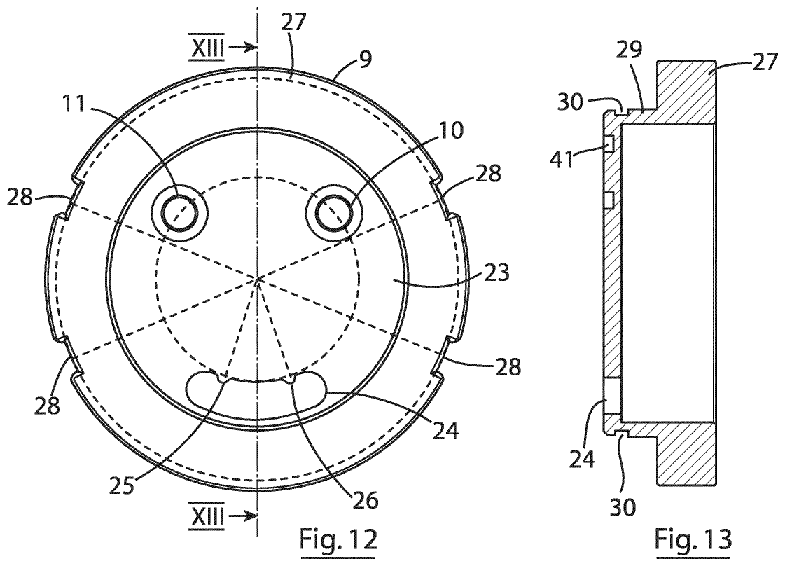

As shown particularly in FIGS. 9 to 13, the rotatable circular dial 9 of the seal mechanism 4 has a circular wall 23 defining the arcuately spaced apart dial openings 10,11 and a peripheral arcuate lug slot 24 opposite the dial openings 10,11 for receiving the projecting lug 16 on the damping plate 15. As shall be explained more fully below, the lug slot 24 is rotatably slidable over the lug 16 between the patient and calibration settings by rotating the dial 9 with the lug 16 being held at each setting by respective first and second keepers 25,26 towards each end of the slot 24. The keepers 25,26 also provide haptic feedback to users to confirm that the lug 16 is in the desired position in the slot 24.

The circular wall 23 of the dial 9 is provided with an enlarged peripheral ring 27 for effecting manual rotation of the dial 9. The peripheral ring 27 is provided with four spaced apart circumferential notch-like grips 28 to assist in manual gripping of the dial 9 during rotation. On its demand valve side, the circular wall 23 is provided with a boss 29 defining a groove 30 engageable with a snap ring 13, which will hold the assembly nut 12 securely to the dial 9 but still allow free rotation of the dial. The dial openings 10,11 extend through the circular wall 23 and the boss 29 and are surrounded by circular recesses 31 defined in the boss 29 for receiving seals 10a and 11a. As indicated above, the three ring seals 32 provide an airtight seal around the dial opening 10,11 that is not in use and are housed in annular recesses 41 defined in rotatable dial 9 at the demand valve side of the dial 9. The annular recesses 41 are circumferentially spaced apart in the dial 9.

The peripheral ring 27 is provided with symbols 33,34 indicative of the patient and calibration settings respectively.

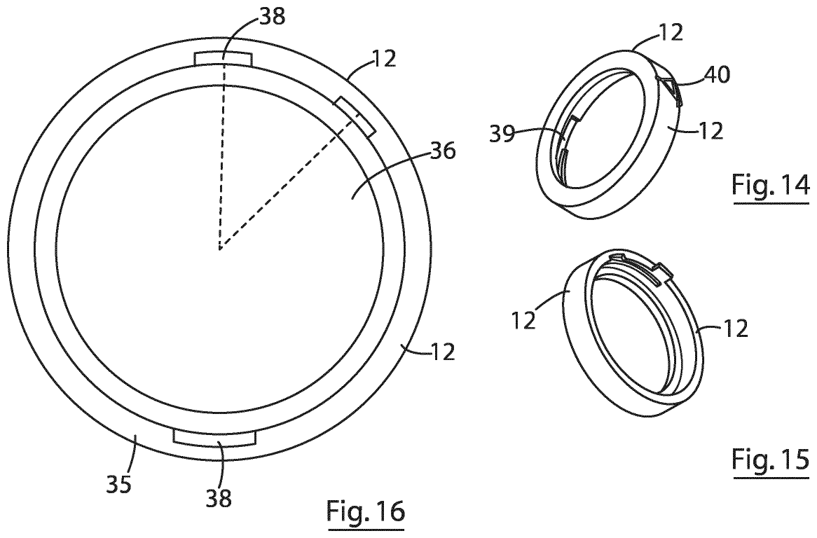

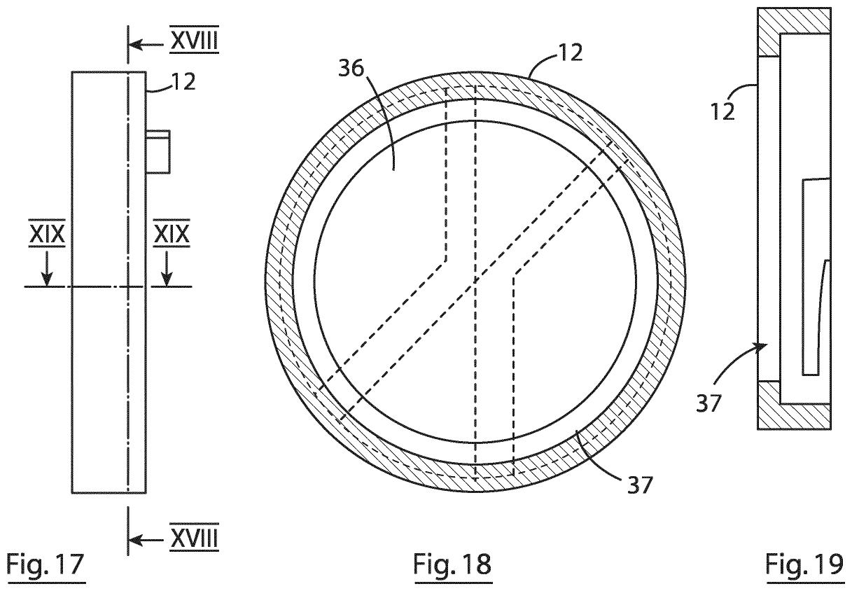

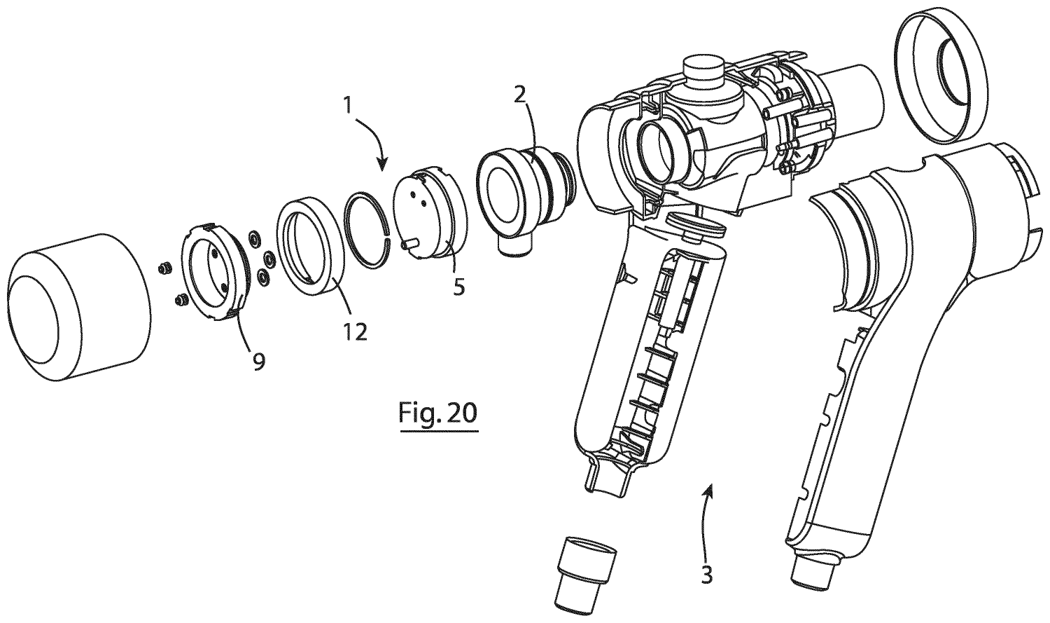

As shown particularly in FIGS. 14 to 19, the assembly nut 12 is made up of an annular ring 35 defining a central opening 36 for mounting the assembly nut 12 over the damping plate 5 at the damping plate shoulder 21 and receiving the boss 29 of the dial 9. The assembly nut 12 is provided with an inner groove 37 surrounding the central opening complementary to and engageable with the snap ring 13 in groove 30 on the dial 9. Circumferentially spaced finger slots 38 on the annular ring 35 are also complementary with the peripheral first and second fingers 18,19 of the securing mechanism 17 defined between the assembly nut 12 and the damping plate 5 for securing the assembly nut 12 to the damping plate 5. An assembly nut catch 39 adjacent to the finger slots 38 also engages the peripheral first and second fingers 18,19 of the securing mechanism on the damping plate 5. The catch 20 on the damping plate sidewall 15 holds the assembly nut 12 (and hence the dial 9) in place on the damping plate 5. In addition a mirror feature of catch 20 can be added, with the function that it stops the nut 12 from moving once it slides over catch 20.

A position indicator 40 is provided on the annular ring to line up with the patient and calibrations symbols 33,34 on the peripheral ring 27 of the seal mechanism 4.

In use, the restrictor 1 of the invention is assembled as previously described and can be retrofitted to the demand valve 2 of the respiratory device 3 via the demand valve mounting 22 or incorporated on the demand valve 2 during manufacture of the respiratory device 3. Where a calibration with a syringe is to be performed, a user rotates the dial 9 to move the seal mechanism 4 into the calibration setting i.e. the relatively smaller calibration opening 6 on the flow plate 5 is in gaseous communication with the atmospheric side of the demand valve 2 so that air is damped to reduce pulses during calibration for improved accuracy. Conversely, where a patient is breathing through the respiratory device 3, the dial 9 is rotated to the patient setting so that the relatively larger respiration opening 7 is in gaseous communication with the atmospheric side of the demand valve 2 so that the patient can breathe with ease on demand. In the calibration setting the relatively smaller calibration opening 6 of the damping plate 5 is contiguous with the first complementary dial opening 10 for a damped reduced pulse flow while, in the patient setting, the relatively larger respiration opening 7 of the damping plate 5 is contiguous with the second complementary dial opening 11 to allow patient breathing on demand.

In the specification the terms “comprise, comprises, comprised and comprising” or any variation thereof and the terms include, includes, included and including” or any variation thereof are considered to be totally interchangeable and they should all be afforded the widest possible interpretation and vice versa.

The invention is not limited to the embodiments hereinbefore described but may be varied in both construction and detail.

1. A demand valve air-flow restrictor co-operable with a demand valve for respiratory devices comprising:
a damping plate having a respiration opening and a calibration opening for controlled air flow through the restrictor and
a seal mechanism attached to the damping plate, the seal mechanism being movable between a patient setting allowing uninterrupted air flow through the respiration opening and a calibration setting allowing damped air flow through the calibration opening.

2. The demand valve restrictor as claimed in claim 1 wherein the respiration opening is a relatively larger opening and the calibration opening is a relatively smaller opening.

3. The demand valve restrictor as claimed in claim 1 wherein the seal mechanism comprises a controller for controlling movement of the seal mechanism between the patient setting and the calibration setting.

4. The demand valve restrictor as claimed in claim 3 wherein the controller comprises a rotatable dial mounted on the damping plate.

5. The demand valve restrictor as claimed in claim 4 wherein the rotatable dial comprises a first dial opening complementary with the respiration opening and a second dial opening complementary with the calibration opening, the first dial opening being contiguous with the respiration opening in the patient setting and the second dial opening being contiguous with the calibration opening in the calibration setting.

6. The demand valve restrictor as claimed in claim 4 wherein the controller further comprises a lug on the damping plate engageable in a slot on the rotatable dial.

7. The demand valve restrictor as claimed in claim 6 wherein the slot comprises keepers to hold the lug at the patient and calibration settings.

8. The demand valve restrictor as claimed in claim 4 further comprising an assembly nut mounted between the damping plate and the dial.

9. The demand valve restrictor as claimed in claim 8 further comprising a securing mechanism between the damping plate and the assembly nut to secure the assembly nut to the damping plate.

10. The demand valve restrictor as claimed in claim 9 wherein the securing mechanism comprises a key-like securing mechanism.

11. The demand valve restrictor as claimed in claim 10 wherein the key-like securing mechanism comprises a catch on the damping plate and a complementary catch on the assembly nut.

12. The demand valve restrictor as claimed in claim 10 wherein the securing mechanism comprises at least one finger on the damping plate and at least one corresponding finger slot on the assembly nut.

13. The demand valve restrictor as claimed in claim 8 wherein the dial comprises a groove engageable with a snap ring which interferes with a shoulder on the assembly nut, to rotatably attach the dial to the assembly nut.

14. The demand valve restrictor as claimed in claim 8 further comprising seals between the damping plate and the seal mechanism to ensure an airtight fit of the seal mechanism on the damping plate.

15. The demand valve restrictor as claimed in claim 1 wherein the demand valve restrictor comprises a demand valve mounting for mounting the restrictor on a demand valve flowhead.

16. The demand valve restrictor as claimed in claim 15 wherein the demand valve mounting is provided on the damping plate.

17. The demand valve restrictor of claim 1 wherein said demand valve restrictor is configured as a respiratory device demand valve.
